(19) United States
(12) Patent Application Publication
(10) Pub. No.: US 2003/0022146 A1
(43) Pub. Date: Jan. 30, 2003

(54) Oxidized bioprosthetic materials

(76) Inventors: Crystal M. Cunanan, Mission Viejo, CA (US); Lillian J. Quintero, Chester, NY (US); Angela de la Fuente, Mission Viejo, CA (US); Patrice Tremble, Santa Rosa, CA (US)

Correspondence Address: Edwards Lifesciences LLC, Law Dept., One Edwards Way, Irvine, CA, 92614, US

(21) Appl. No.: 09/915,489

(22) Filed: Jul. 26, 2001

(51) Int. Cl. A01N 1/02; A61F 2/00

(52) U.S. Cl. 435/1.1; 623/66.1

(57) ABSTRACT

A method for chemical fixation of tissues by exposing the tissue to a chemical fixative agent, under oxidative conditions. The chemical fixative agents useable in this method include aldehydes (e.g., formaldehyde, glutaraldehyde, dialdehyde starch), isocyanates (e.g., hexamethylene diisocyanate) and certain polyepoxy compounds (e.g., DENACOL). The oxidative conditions may be provided by heating of a chemical fixative solution that contains the crosslinking agent, in the presence of room air or oxygen. Alternatively, the oxidative conditions may be provided by adding one or more oxidizing chemicals (e.g., hydrogen peroxide or other peroxides, sodium periodate or other periodates, diisocyanates, halogens, n-bromosuccinimide or other halogenated compounds, permanganates, ozone, chromic acid, sulfuryl chloride, sulfoxides, selenoxides, etc.) to the chemical fixative solution. Alternatively, the oxidative conditions may be provided by irradiation (e.g., alpha, beta, ultraviolet, electron beam, gamma rays) of the fixative solution in the presence of room air or oxygen.

### FIELD OF THE INVENTION

This invention pertains generally to medical devices & methods and more particularly to implantable bioprosthetic materials and their methods of manufacture.

### BACKGROUND OF THE INVENTION

I. The Preparation and Use of Bioprosthetic Devices:

Implantable bioprosthetic devices are formed, wholly or partially, of human or animal tissue that has been preserved by freezing (i.e., cryopreservation) or by chemical fixation (i.e., tanning). The types of biological tissues used as bioprostheses include cardiac valves, blood vessels, skin, dura mater, pericardium, ligaments and tendons. These biological tissues typically contain connective tissue proteins (i.e., collagen and elastin) that act as the supportive framework of the tissue. The pliability or rigidity of each biological tissue is largely determined by the relative amounts of collagen and elastin present within the tissue and/or by the physical structure and confirmation of its connective tissue framework. Collagen is the most abundant connective tissue protein present in most tissues. Each collagen molecule is made up of three (3) polypeptide chains intertwined in a coiled helical configuration.

The techniques used for chemical fixation of biological tissues typically involve the exposure of the biological tissue to one or more chemical fixatives (i.e., tanning agents) that form cross-linkages between the polypeptide chains within a given collagen molecule (i.e., intramolecular crosslinkages), or between adjacent collagen molecules (i.e., intermolecular crosslinkages).

Examples of chemical fixative agents that have heretofore been utilized to cross-link collagenous biological tissues include; formaldehyde, glutaraldehyde, dialdehyde starch, hexamethylene diisocyanate and certain polyepoxy compounds. Of the various chemical fixatives available, glutaraldehyde is the most widely used.

Glutaraldehyde is used as the fixative for many commercially available bioprosthetic products, such as porcine bioprosthetic heart valves (e.g., the Carpentier-Edwards® Stented Porcine Bioprosthesis), bovine pericardial heart valve prostheses (e.g., Carpentier-Edwards® PERIMOUNT® Pericardial Bioprosthesis) and stentless porcine aortic prostheses (e.g., Edwards PRIMA Plus™ Stentless Aortic Bioprosthesis), all available from Edwards Lifesciences, Irvine, Calif. 92614.

Polyepoxy compounds that have heretofore been known for use as collagen crosslinking agents are described in U.S. Pat. No. 4,806,959 (Noishiki et al.) and U.S. Pat. No. 5,080,670 (Imamura et al.). At least some of these heretofore-known polyepoxy fixatives are commercially available under the trademark DENACOL from Nagase Chemicals, Ltd., Osaka, Japan. In particular, one difunctional epoxy compound which has been disclosed for use as a collagen cross linking agent is an ethylene glycol diglycidyl ether based compound commercially available from Nagase Chemicals, Ltd. of Osaka, Japan under the designation DENACOL.

ii. The Use of Implantable Scaffolds for Tissue Engineering:

Tissue engineering is an emerging technology that employs principles of biology and engineering to develop viable “engineered tissue” for restoration, replacement, maintenance or improvement of human organs or tissues. In most applications, the engineered tissue becomes permanently integrated within the patient, thereby affording a potentially permanent cure for an offending disease or deformity.

Although cells have been cultured in vivo for many years, it is only recently that advancements in the field of tissue engineering have made it possible to grow cells in the particular three-dimensional structure needed for repair or reconstruction of an organ or anatomical structure. Three general approaches to tissue engineering have been elucidated:

1. Designing and growing of human tissues outside the body, for later surgical implantation. The most common example of this is skin grafts used for treatment of bums. In some instances, it is desirable to provide a preformed “scaffolding” device (i.e., a specifically configured matrix) to cause the cultured cells to grow into the desired shape or configuration. One such method for ex vivo culturing of oral and dental tissues on a structural matrix is described in U.S. Pat. No. 5,885,829 (Mooney, et al.) entitled Engineering Oral Tissues.

2. Implanting a cell-containing or cell-free scaffolding device (i.e., matrix) that induces regeneration of tissues within the body. Certain biological agents or “signal” molecules, like growth factors, may be administered to assist in the scaffold-guided tissue regeneration. To date, polymeric materials have been used to form the scaffolding devices to which cells attach and grow to reconstitute tissues. One example of this approach is the implantation of a biomaterial scaffold to promote bone regrowth in patients who suffer from periodontal disease (i.e., chronic gum disease).

3. Implanting or attaching internal or external devices that contain functional tissues, to replace the function of diseased internal tissues. This approach involves isolating cells from the patient's body, placing the cells on or within a scaffolding device (i.e., a structural matrix), and then implanting the cell-impregnated scaffold device inside the body, or attaching it to the body. Examples of this approach include the implantation of vascular grafts that have been lined or seeded with the patient's own endothelial cells and the implantation of artificial livers.

Ongoing research in the area of tissue engineering is attempting to engineer skin, cartilage, bone, central nervous system tissues, muscle, liver, and pancreatic islet (insulin-producing) cells. Tissue engineering techniques may one day enable organ transplants to be conducted using engineered tissues or organs that originated from the patient's own body, thereby eliminating the potential for transplant rejection and the need for anti-rejection therapies such as the long term administration of immunosuppressive drugs.

To date most of the scaffolds or matrices used in tissue engineering have been made of synthetic polymer materials. Although it would be desirable to use natural tissues as scaffolds, most natural tissues fixed by the tissue fixation techniques of the prior art have failed to exhibit the durability required for use in such scaffolding applications.

There presently remains a need in the art for the development of new methods for fixing (i.e., tanning) biological tissues to provide bioprosthetic tissues that are biologically compatible and durable enough for use in various applications including scaffolding applications in tissue engineering.

### SUMMARY OF THE INVENTION

Broadly stated, the present invention provides a method for chemical treatment of tissues by exposing the tissue to a solution under oxidative conditions. The solution may be a chemical fixative agent including aldehydes (e.g., formaldehyde, glutaraldehyde, dialdehyde starch), isocyanates (e.g., hexamethylene diisocyanate) and certain polyepoxy compounds (e.g., DENACOL). The oxidative conditions may be provided by heating of the solution that contains a crosslinking agent, in the presence of room air or oxygen. Alternatively, the oxidative conditions may be provided by one or more oxidizing chemicals (e.g., hydrogen peroxide or other peroxides, sodium periodate or other periodates, diisocyanates, halogens, n-bromosuccinimide or other halogenated compounds, permanganates, ozone, chromic acid, sulfuryl chloride, sulfoxides, selenoxides, etc.), or adding such chemicals to a chemical fixative solution. Alternatively, the oxidative conditions may be provided by irradiation (e.g., alpha, beta, ultraviolet, electron beam, gamma rays) of the solution in the presence of room air or oxygen. Tissues fixed under oxidative conditions in accordance with this invention exhibit improved resistance to acid hydrolysis, and thus are likely to exhibit improved stability when compared to tissues fixed in the absence of oxidative conditions.

The solution may be a fixative such as glutaraldehyde or Denacol, or may be peroxide. An exemplary method according to the invention involves exposing the tissue to oxidative conditions by placing the tissue in a solution containing 0.2-2.0% glutaraldehyde, maintaining the glutaraldehyde solution at 25-70° C. for a period of 0.5-60 days; and, removing the tissue from the glutaraldehyde solution. The solution desirably has a glutaraldehyde concentration of about 0.625%, and is maintained at about 45-55° C. for a period of between about 7 to 14 days, and preferably closer to 7 days.

Further in accordance with the invention, there are provided bioprosthetic devices or articles that are formed wholly or partially of tissue prepared by the above-summarized method of the present invention. Examples of specific biological tissues which may be utilized to prepare bioprosthetic devices or articles in accordance with this invention include, but are not necessarily limited to: heart valves; venous valves; blood vessels; ureter; tendon; dura mater; skin; pericardium; cartilage (e.g., meniscus); ligament; bone; intestine (e.g., intestinal wall); and periostium.

Further in accordance with the present invention, there are provided methods for treatment of diseases and disorders of mammalian patients, by implanting bioprosthetic devices that have been prepared by the oxidative fixation methods of the present invention.

Such treatment methods include, but are not limited to, a) the surgical replacement of diseased heart valves with bioprosthetic heart valves prepared by the fixation method of this invention, b) the repair or bypassing of blood vessels by implanting vascular grafts prepared by the fixation method of this invention, c) the surgical replacement or repair of torn or deficient ligaments by implanting bioprosthetic ligaments prepared by the fixation method of this invention, and, d) the repair, reconstruction, reformation, enhancement, bulking, ingrowth, reconstruction or regeneration of native tissues by implanting one or more bioprosthetic tissue scaffolds that have been prepared by the fixation method of this invention (e.g., tissue engineering with a natural tissue scaffold).

Further aspects and objects of the present invention will become apparent to those skilled in the relevant art, upon reading and understanding the “Detailed Description of Exemplary Embodiments” set forth herebelow.

### BRIEF DESCRIPTION OF THE DRAWINGS

FIG. 1 is a general flow diagram of an oxidative treatment method of the present invention.

FIG. 2 is a flow diagram of an oxidative treatment method of the present invention, wherein the oxidative conditions are provided by heating of flowing solution in the presence of oxygen.

Additional embodiments and aspects of the invention may become apparent to those of skill in the art upon reading and understanding of the detailed description and specific examples set forth herebelow.

### DETAILED DESCRIPTION OF EXEMPLARY EMBODIMENTS

The following examples are provided for the purpose of describing and illustrating a few exemplary embodiments of the invention only. Other embodiments of the invention are possible, but are not described in detail here. Thus, these examples are not intended to limit the scope of the invention in any way.

Those of skill in the art will appreciate that oxidative conditions may be created in various ways, ranging from simple heating of the fixation solution in the presence of oxygen (e.g., heating the solution while blanketed with room air or oxygen or while bubbling room air or oxygen through the fixative solution) to adding oxidative chemicals to the fixative solution (e.g., adding liquid hydrogen peroxide solution or bubbling gaseous ozone through the fixative solution).

1. General Methodology

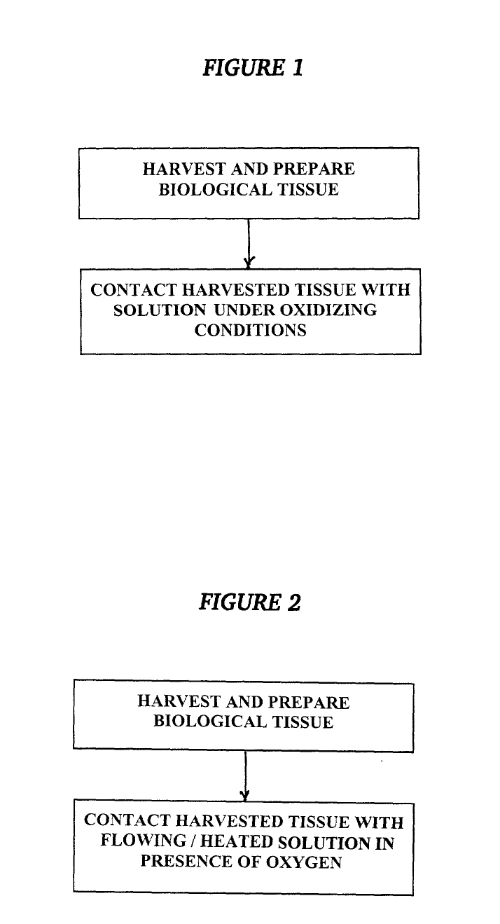

FIG. 1 is a flow diagram setting forth the general method of preparing a bioprosthetic material in accordance with the present invention. As shown, the method comprises a) harvesting a desired biological tissue from a human or animal donor and b) exposing the tissue to at least one fixative agent under oxidative conditions. The fixative agent may be any suitable chemical that crosslinks connective tissue proteins, such as:

an aldehyde (e.g., formaldehyde, glutaraldehyde, dialdehyde starch);

an isocyanate (e.g., hexamethylene diisocyanate); and/or

a polyepoxy compound (e.g., a polyglycidyl ether).

The oxidative conditions may be provided by heating of a chemical fixative solution that contains the crosslinking agent, in the presence of room air or oxygen. Alternatively, the oxidative conditions may be provided by adding one or more oxidizing chemicals (e.g., hydrogen peroxide or other peroxides, sodium periodate or other periodates, diisocyanates, halogens, n-bromosuccinimide or other halogenated compounds, permanganates, ozone, chromic acid, sulfuryl chloride, sulfoxides, selenoxides, etc.) to the chemical fixative solution. Alternatively, the oxidative conditions may be provided by any suitable means for promoting chemical oxidation including:

heating of the fixative during exposure of the biological tissue, in the presence of oxygen (e.g., ambient oxygen, room air, gaseous oxygen);

irradiation (e.g., alpha, beta, ultraviolet, electron beam, gamma rays) of the fixative during exposure of the biological tissue, in the presence of oxygen (e.g., ambient oxygen, room air, gaseous oxygen); and/or

addition of a chemical oxidation agent (e.g., a peroxide, periodate, permanganate, ozone, n-bromosuccinimide, halogens, etc.) to the fixative prior to or during exposure of the biological tissue.

FIG. 2 shows an example of a method wherein heat is used to provide the oxidative conditions during fixation of a biological tissue. The particular steps of this method are as follows:

2. A Method Where Oxidation is Achieved by Heating of the Fixative

The flow diagram of FIG. 2 shows an example of a tissue fixation method wherein oxidative conditions are created by heating of the fixative (e.g., glutaraldehyde) during exposure of the tissue. The steps of the method shown in FIG. 2 are as follows:

### Step 1: Harvest/Prepare Biological Tissue

The desired biological tissue is harvested (i.e., surgically removed or cut away from its host animal). Thereafter, the tissue is typically trimmed or cut to size and washed with sterile water, basic salt solution, saline or other suitable washing solution.

### Step 2: Fix Biological Tissue With Heated/Flowing Fixative Solution in the Presence of Room Air or Oxygen

The biological tissue is then placed in a circulating fixation column of the type described in U.S. Pat. No. 5,931,969, which is hereby expressly incorporated by reference. The fixation column is filled to an appropriate level with an aqueous solution of 0.625% by weight glutaraldehyde buffered to a pH of approximately 7.4 by a suitable buffer such as a phosphate buffer. Room air is allowed to blanket or cover the glutaraldehyde solution. An immersible heater is positioned in the glutaraldehyde solution and the solution is circulated through the column type device. The previously harvested, trimmed and washed tissue is then positioned in the column device as described in U.S. Pat. No. 5,931,969, the glutaraldehyde solution is circulated through the device and the heater is used to maintain the temperature of the glutaraldehyde solution at 50+/−5 degrees C. for a period of between about 7 to 14 days. Thereafter, the tissue is removed from the column and rinsed.

It will be appreciated that, instead of placing the tissue in a fixation column device of the type described above, any other suitable means of causing the fixative solution to move or flow may also be used. For example, the tissue may be placed in the fixative solution and the solution may then be shaken, stirred, or otherwise agitated using any of the numerous types of shakers and stirrers known in the art, including the shakers and stirrers shown in U.S. Pat. No. 5,931,969.

When tissues fixed by this method are immersed in 6N Hydrochloric acid at 110 degrees C. for 5 days, they exhibit minimal degradation. In contrast, tissues fixed by traditional glutaraldehyde fixation techniques typically exhibit substantial degradation after less than 24 hours exposure to 6N Hydrochloric acid at 110 degrees C.

While the foregoing describes the preferred embodiments of the invention, various alternatives, modifications, and equivalents may be used. Moreover, it will be obvious that certain other modifications may be practiced within the scope of the appended claims.

What is claimed is:

1. A method for chemical treatment of biological tissue, comprising the steps of:
(a) providing a quantity of biological tissue which contains connective tissue protein; and,
(b) contacting the biological tissue with a solution under oxidizing conditions for sufficient time to impart enhanced stability compared to traditional means of fixing connective tissue protein within the biological tissue.

2. A method according to claim 1 wherein the oxidizing conditions are provided in step (b) by heating the solution in the presence of oxygen.

3. A method according to claim 2 wherein the presence of oxygen is provided by ambient oxygen in the solution.

4. A method according to claim 2 wherein at least some of the oxygen present is provided by allowing the solution to contact atmospheric air, oxygen or an oxygen-containing gas solution.

5. A method according to claim 2 wherein at least some of the oxygen present is provided by bubbling oxygen or an oxygen-containing gas mixture through the solution.

6. A method according to claim 1 wherein the oxidizing conditions are provided in step (b) by combining an oxidizing agent with the solution in the presence of oxygen.

7. A method according to claim 6 wherein the oxidizing agent is selected from the group of oxidizing agents consisting of a peroxide, a compound containing peroxide, hydrogen peroxide, a periodate, a compound containing periodate, sodium periodate, a diisocyanate compound, a halogen, a compound containing halogen, n-bromosuccinimide, a permanganate, a compound containing permanganate, ozone, a compound containing ozone, chromic acid, sulfuryl chloride, a sulfoxide, a selenoxide, and combinations thereof.

8. A method according to claim 6 wherein the presence of oxygen is provided by ambient oxygen in the solution.

9. A method according to claim 6 wherein at least some of the oxygen present is provided by allowing the solution-oxidizing agent mixture to contact atmospheric air, oxygen or an oxygen-containing gas mixture.

10. A method according to claim 6 wherein at least some of the oxygen present is provided by bubbling oxygen or an oxygen-containing gas mixture through the solution.

11. A method according to claim 1 wherein the oxidizing conditions are provided in step (b) by irradiating the solution in the presence of oxygen.

12. A method according to claim 11 wherein the solution is irradiated by a type of radiation energy selected from the group of alpha ionizing radiation, beta ionizing radiation, ultraviolet radiation, electron beam radiation, gamma rays, and combinations thereof.

13. A method according to claim 11 wherein the presence of oxygen is provided by ambient oxygen in the solution.

14. A method according to claim 11 wherein at least some of the oxygen present is provided by allowing the solution to contact atmospheric air, oxygen or an oxygen-containing gas mixture.

15. A method according to claim 11 wherein at least some of the oxygen present is provided by bubbling oxygen or an oxygen-containing gas mixture through the solution.

16. A method according to claim 1 wherein the solution is flowing.

17. A method according to claim 16 wherein the flowing of the solution is effected by placing the solution and the tissue in a container, wherein the solution is heated and circulated through the container.

18. A method according to claim 1, wherein step (b) comprises the steps of:
placing the tissue in a solution containing 0.2-2.0% glutaraldehyde;
maintaining the glutaraldehyde solution at 25-70° C. for a period of 0.5-60 days; and,
removing the tissue from the glutaraldehyde solution.

19. A method according to claim 18 wherein the solution has a glutaraldehyde concentration of about 0.625%.

20. A method according to claim 19 wherein the 0.625% glutaraldehyde solution is maintained at about 45-55° C. for a period of between about 7 and 14 days.

21. A method according to claim 1 wherein the solution is a fixative.

22. A method according to claim 21 wherein fixative is glutaraldehyde.

23. A method according to claim 21 wherein fixative is Denacol.

24. A method according to claim 1 wherein the solution is peroxide.

25. A bioprosthesis comprising tissue that has been prepared by a method comprising the steps of:
(a) providing a quantity of biological tissue which contains connective tissue protein; and,
(b) contacting the biological tissue with a solution under oxidizing conditions for sufficient time to result in crosslinking of connective tissue protein within the biological tissue.

26. A bioprosthesis according to claim 25 wherein the oxidizing conditions in step (b) are provided by heating the solution in the presence of oxygen.

27. A bioprosthesis according to claim 25 wherein the presence of oxygen in step (b) is provided by ambient oxygen in the solution.

28. A bioprosthesis according to claim 25 wherein at least some of the oxygen present is provided by allowing the solution to contact atmospheric air, oxygen or an oxygen-containing gas solution.

29. A bioprosthesis according to claim 25 wherein at least some of the oxygen present in step (b) is provided by bubbling oxygen or an oxygen-containing gas mixture through the solution.

30. A bioprosthesis according to claim 25 wherein the oxidizing conditions are provided in step (b) by combining an oxidizing agent with the solution in the presence of oxygen.

31. A bioprosthesis according to claim 30 wherein the oxidizing agent is selected from the group of oxidizing agents consisting of a peroxide, a compound containing peroxide, hydrogen peroxide, a periodate, a compound containing periodate, sodium periodate, a diisocyanate compound, a halogen, a compound containing halogen, n-bromosuccinimide, a permanganate, a compound containing permanganate, ozone, a compound containing ozone, chromic acid, sulfuryl chloride, a sulfoxide, a selenoxide, and combinations thereof.

32. A bioprosthesis according to claim 30 wherein the presence of oxygen is provided by ambient oxygen in the solution-oxidizing agent mixture.

33. A bioprosthesis according to claim 30 wherein at least some of the oxygen present is provided by allowing the solution-oxidizing agent mixture to contact atmospheric air, oxygen or an oxygen-containing gas mixture.

34. A bioprosthesis according to claim 30 wherein at least some of the oxygen present is provided by bubbling oxygen or an oxygen-containing gas mixture through the solution.

35. A bioprosthesis according to claim 25 wherein the oxidizing conditions are provided in step (b) by irradiating the solution in the presence of oxygen.

36. A bioprosthesis according to claim 35 wherein the solution is irradiated by a type of radiation energy selected from the group consisting of: alpha ionizing radiation, beta ionizing radiation, ultraviolet radiation, electron beam radiation, gamma rays, and combinations thereof.

37. A bioprosthesis according to claim 35 wherein the presence of oxygen is provided by ambient oxygen in the solution.

38. A bioprosthesis according to claim 35 wherein at least some of the oxygen present is provided by allowing the solution to contact atmospheric air, oxygen or an oxygen-containing gas mixture.

39. A bioprosthesis according to claim 35 wherein at least some of the oxygen present is provided by bubbling oxygen or an oxygen-containing gas mixture through the solution.

40. A bioprosthesis according to claim 25 wherein the solution is flowing.

41. A bioprosthesis according to claim 40 wherein the flowing of the solution is effected by placing the solution and the tissue in a fixation container, wherein the solution is heated and circulated through the container.
